(19) United States
(12) Patent Application Publication
(10) Pub. No.: US 2024/0065702 A1
(43) Pub. Date: Feb. 29, 2024

(54) EMBOLIZATION DEVICE

(71) Applicant: KANEKA CORPORATION, Osaka-shi, Osaka (JP)

(72) Inventors: Atsushi OGAWA, Tokyo (JP); Masayuki TAKATERA, Settsu-shi (JP)

(73) Assignee: KANEKA CORPORATION, Osaka-shi, Osaka (JP)

(21) Appl. No.: 18/268,443

(22) PCT Filed: Dec. 15, 2021
(86) PCT No.: PCT/JP2021/046329

§ 371 Date: Jun. 20, 2023

(30) Foreign Application Priority Data
Dec. 23, 2020 (JP) 2020-213880

(51) Int. Cl. A61B 17/12; A61B 17/00

(52) U.S. Cl. CPC A61B 17/12177; A61B 17/00234; A61B 17/12113; A61B 2017/00292; A61B 2017/00681; A61B 2017/00867; A61B 2017/00982; A61B 2017/12068

(57) ABSTRACT

An embolization device for a bump in a lumen, comprising: an outer tube having a distal end and a proximal end; a basket disposed in a lumen of the outer tube, having a plurality of wires, expandable when coming out of the outer tube, and composed of a mesh-like wall surface on which the plurality of wires intersect; and a basket pusher disposed on a proximal side of the basket, wherein the basket has a first bundling portion at which the plurality of wires are bundled and fixed on a distal side thereof and a second bundling portion at which the plurality of wires are bundled and fixed on the proximal side thereof, the plurality of wires exist inside the second bundling portion, and no passage for inserting a medical long object into the basket exists inside the second bundling portion.

### TECHNICAL FIELD

The present invention relates to an embolization device for forming emboli in blood vessels in vascular disease-affected areas.

### BACKGROUND ART

Endovascular treatment is one of the treatment methods for vascular lesions such as head and neck aneurysms, arteriovenous malformations, arteriovenous fistulas, pulmonary vascular malformations, renal vascular malformations, renal arteries, and abdominal aneurysms. As an endovascular treatment, for example, embolization, in which an embolization device having an embolization coil is indwelled at a target site such as the inside of a bump to promote formation of an embolus, thereby preventing rupture of an aneurysm, is used.

When the opening of an aneurysm or the like in a vessel wall is large, an embolization coil indwelled inside the aneurysm may come out of the aneurysm. In embolization, an indwelling device to be placed in an aneurysm or in a blood vessel near the opening of the aneurysm may be used in order to prevent deviation of a coil indwelled inside an aneurysm.

For example, Patent Literature 1 describes an aneurysm occlusion device including a bulbous body portion sized to be received within an aneurysm and an anchor, wherein the body portion and the anchor are integrally formed of a resilient fabric permitting the occlusion device to be collapsed for deployment and resiliently self-expand to occlude the aneurysm. Patent Literature 2 describes a device for treating an aneurysm, the device having an expandable frame which is folded when delivered, expands so as to bridge a neck of the aneurysm and is brought into a contact with an inner wall of the aneurysm when deployed, and is covered with a mesh that is permeable to blood flow, the device being configured such that an embolic agent is injected through the permeable mesh or a valve after the frame is deployed in the aneurysm. Patent Literature 3 describes a device for the treatment of a vascular defect, the device including: at least one occlusive member having a first unexpanded configuration and a second expanded configuration; and a securement member for securing the device and having an unexpanded configuration in which the securement member can pass through an opening of a support structure and an expanded state in which the securement member cannot pass through the opening of the support structure. Patent Literature 4 describes an implantable retainer deliverable via an elongated tubular delivery device for retaining a vaso-occlusive device in an aneurysm, the retainer including: a core wire having a proximal end and a distal end; a joint electrolytically severable upon an application of a suitable current to the joint; a retainer assembly; and a plurality of external array elements configured to remain outside the aneurysm when the retainer assembly is deployed. Patent Literature 5 describes an occlusion device suitable for endovascular treatment of an aneurysm in a blood vessel in a patient, the occlusion device including: a substantially tubular structure having a first expanded condition in which the structure has an exterior surface capable of contacting the aneurysm and a second collapsed condition in which the structure has dimensions suitable for insertion through the vasculature of the patient and through a neck of the aneurysm; and a control ring having a substantially annular body to prevent radial expansion of the structure.

### CITATION LIST

### Patent Literature

PATENT LITERATURE 1: WO 1997/026939
PATENT LITERATURE 2: JP 2001-518320 T
PATENT LITERATURE 3: JP 2005-537092 T
PATENT LITERATURE 4: JP 2008-173497 A
PATENT LITERATURE 5: JP 2015-196092 A
PATENT LITERATURE 6: JP 2004-154536 A
PATENT LITERATURE 7: JP 2018-69092 A
PATENT LITERATURE 8: JP 2020-503147 T

### SUMMARY OF INVENTION

### Technical Problem

However, in the indwelling devices as in Patent Literatures 1 to 5, an opening through which a medical long object such as a coil can be inserted into the indwelling device is limited. Therefore, depending on the condition of a lesion site or a procedure, it may be difficult to insert the medical long object into the indwelling device. In the indwelling devices as in Patent Literatures 1 to 5, a problem that it is difficult to indwell the coil in a bump, a problem that the size of the indwelling device itself is increased in order to facilitate insertion of the coil into the indwelling device, or a problem that the coil in the indwelling device indwelled in a bump passes through the opening or the like of the indwelling device and comes out of the bump, may arise, so that there is room for improvement.

In addition, in the indwelling devices as in Patent Literatures 6 to 8, in order to stably place a medical long object such as a coil in a bump, an assist stent may be placed in a blood vessel facing the bump. When the assist stent is placed in the blood vessel, there is a problem that it is necessary to use a medical drug such as an antiplatelet agent over a long period of time. The present invention eliminates the need for the placement of the assist stent by realizing the role of the assist stent to be placed in a blood vessel by a device to be placed in a bump. As a result, the use of a medical drug such as an antiplatelet agent is reduced.

The present invention has been made in view of the above circumstances, and an object of the present invention is to provide an embolization device that makes it easier to insert a medical long object into a basket and makes it difficult for the medical long object placed inside the basket to come out of the basket.

### Solution to Problem

An embolization device, which solves the above problem, comprises the embolization device for a bump in a lumen, comprising: an outer tube having a distal end and a proximal end; a basket disposed in a lumen of the outer tube, having a plurality of wires, expandable when coming out of the outer tube, and composed of a mesh-like wall surface on which the plurality of wires intersect; and a basket pusher disposed on a proximal side of the basket, wherein the basket has a first bundling portion at which the plurality of wires are bundled and fixed on a distal side thereof and a second bundling portion at which the plurality of wires are bundled and fixed on the proximal side thereof, the plurality of wires exist inside the second bundling portion, and no passage for inserting a medical long object in a direction from the proximal side toward the distal side of the basket exists inside the second bundling portion.

In the embolization device of the present invention, it is preferable that an intersection point of the wires of the mesh of the basket is not fixed.

In the embolization device of the present invention, it is preferable that a position of the intersection point of the wires of the mesh of the basket is shifted by an external force and a shape of an opening of the mesh is changed.

In the embolization device of the present invention, it is preferable that in a state where no external force is applied to the basket, the basket viewed in a direction perpendicular to a longitudinal direction of the outer tube has an opening of the mesh in which an angle formed on the distal side between a straight line passing through the first bundling portion and the second bundling portion and the wire at a midpoint between the first bundling portion and the second bundling portion is equal to or larger than 30 degrees and smaller than 90 degrees.

In the embolization device of the present invention, it is preferable that in a state where no external force is applied to the basket, the basket viewed in the direction perpendicular to the longitudinal direction of the outer tube has an opening of the mesh in which an angle in an axial direction at the intersection point of the wires is not smaller than 60 degrees and not larger than 160 degrees.

In the embolization device of the present invention, it is preferable that the basket is deformable and is slidable inside a tube having an inner diameter of 0.021 inch or smaller.

In the embolization device of the present invention, it is preferable that further comprising a severable connection member disposed on the proximal side with respect to the basket and on the distal side with respect to the basket pusher.

In the embolization device of the present invention, it is preferable that a material forming the connection member has a property of melting due to heat, and the embolization device has a heating mechanism for heating the connection member.

In the embolization device of the present invention, it is preferable that in a cross-section perpendicular to the longitudinal direction of the outer tube, the connection member is disposed inside the second bundling portion, and the wires are disposed outside the connection member.

In the embolization device of the present invention, it is preferable that in a state where no external force is applied to the basket, the first bundling portion is located on the proximal side with respect to a distal end of the basket.

In the embolization device of the present invention, it is preferable that in a state where no external force is applied to the basket, the first bundling portion is located on the distal side with respect to a distal end of the basket.

In the embolization device of the present invention, it is preferable that in a state where no external force is applied to the basket, the second bundling portion is located on the distal side with respect to a proximal end of the basket.

In the embolization device of the present invention, it is preferable that in a state where no external force is applied to the basket, the second bundling portion is located on the proximal side with respect to a proximal end of the basket.

In the embolization device of the present invention, it is preferable that the first bundling portion is located on the proximal side with respect to the distal end of the basket in the lumen of the outer tube.

In the embolization device of the present invention, it is preferable that the second bundling portion is located on the distal side with respect to the proximal end of the basket in the lumen of the outer tube.

In the embolization device of the present invention, it is preferable that at least one of the first bundling portion and the second bundling portion is bundled by a bundling tool.

In the embolization device of the present invention, it is preferable that the wires include a first wire and a second wire, and a material forming the first wire is different from a material forming the second wire.

In the embolization device of the present invention, it is preferable that the second wire contains an X-ray impermeable material.

In the embolization device of the present invention, it is preferable that the second wire is composed of a structure obtained by twisting together a plurality of wire materials.

### Advantageous Effects of Invention

Since the embolization device of the present invention includes the basket composed of the mesh-like wall surface on which the plurality of wires intersect, the basket has the first bundling portion at which the plurality of wires are bundled and fixed on the distal side thereof and the second bundling portion at which the plurality of wires are bundled and fixed on the proximal side thereof, the plurality of wires exist inside the second bundling portion, and no passage for inserting the medical long object in the direction from the proximal side toward the distal side of the basket exists inside the second bundling portion, the medical long object is inserted into the basket from the mesh-like wall surface on which the plurality of wires intersect. A plurality of openings through which the medical long object can be inserted exist in the mesh-like wall surface, and by selecting an appropriate opening of the mesh according to the condition of a lesion site or a procedure, it is made possible to easily indwell the medical long object at the lesion site. In addition, since the basket is composed of the mesh-like wall surface on which the plurality of wires intersect, the mesh of the basket is finer than that of a conventional embolization device. Therefore, it is difficult for the medical long object, such as a coil, that is placed in the basket to pass through the wall surface of the basket, so that it can be difficult for the medical long object placed inside the basket to come out of the basket.

### BRIEF DESCRIPTION OF DRAWINGS

FIG. 1 represents a cross-sectional view, of an embolization device according to an embodiment of the present invention, parallel to the longitudinal direction of an outer tube.

FIG. 2 represents a plan view of a basket of the embolization device shown in FIG. 1.

FIG. 3 represents a cross-sectional view of the embolization device shown in FIG. 1, taken along a line III-III.

FIG. 4 represents a plan view showing an angle of an opening of a mesh of the basket of the embolization device shown in FIG. 1.

FIG. 5 represents a schematic diagram of a basket of an embolization device according to an embodiment of the present invention.

FIG. 6 represents a schematic diagram of a basket of an embolization device according to an embodiment of the present invention.

FIG. 7 represents a schematic diagram of a basket of an embolization device according to an embodiment of the present invention.

FIG. 8 represents a schematic diagram of a basket of an embolization device according to an embodiment of the present invention.

FIG. 9 represents a schematic diagram of a basket of an embolization device according to an embodiment of the present invention.

FIG. 10 represents a schematic diagram of a basket of an embolization device according to an embodiment of the present invention.

FIG. 11 represents a schematic diagram of a basket of an embolization device according to an embodiment of the present invention.

FIG. 12 represents a schematic diagram of a basket of an embolization device according to an embodiment of the present invention.

FIG. 13 represents a schematic diagram of a basket of an embolization device according to an embodiment of the present invention.

FIG. 14 represents a schematic diagram of a state where a basket of an embolization device according to an embodiment of the present invention is placed in a bump at a terminal portion.

FIG. 15 represents a schematic diagram of a state where a medical long object is placed in the basket in the bump shown in FIG. 14.

FIG. 16 represents a schematic diagram of a state where a medical long object and a basket of an embolization device according to an embodiment of the present invention are placed in a bump at a side wall portion.

FIG. 17 represents a schematic diagram of a state where a medical long object and a basket of an embolization device according to an embodiment of the present invention are placed at a main tube peripheral portion.

### DESCRIPTION OF EMBODIMENTS

Hereinafter, the present invention is specifically described below based on the following embodiments; however, the present invention is not restricted by the embodiments described below of course, and can be certainly put into practice after appropriate modifications within in a range meeting the gist of the above and the below, all of which are included in the technical scope of the present invention. In the drawings, hatching or a reference sign for a member may be omitted for convenience, and in such a case, the description and other drawings should be referred to. In addition, sizes of various members in the drawings may differ from the actual sizes thereof, since priority is given to understanding the features of the present invention.

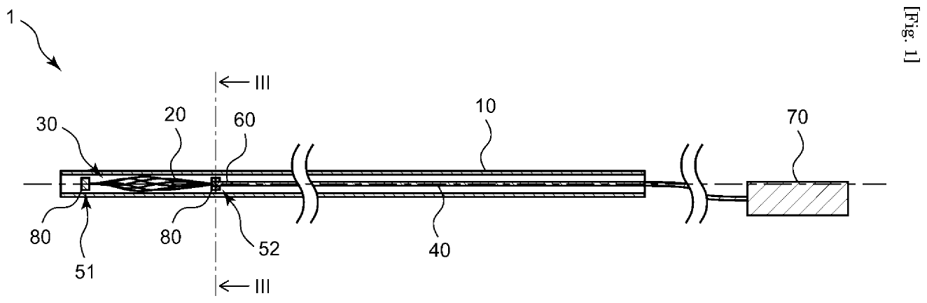

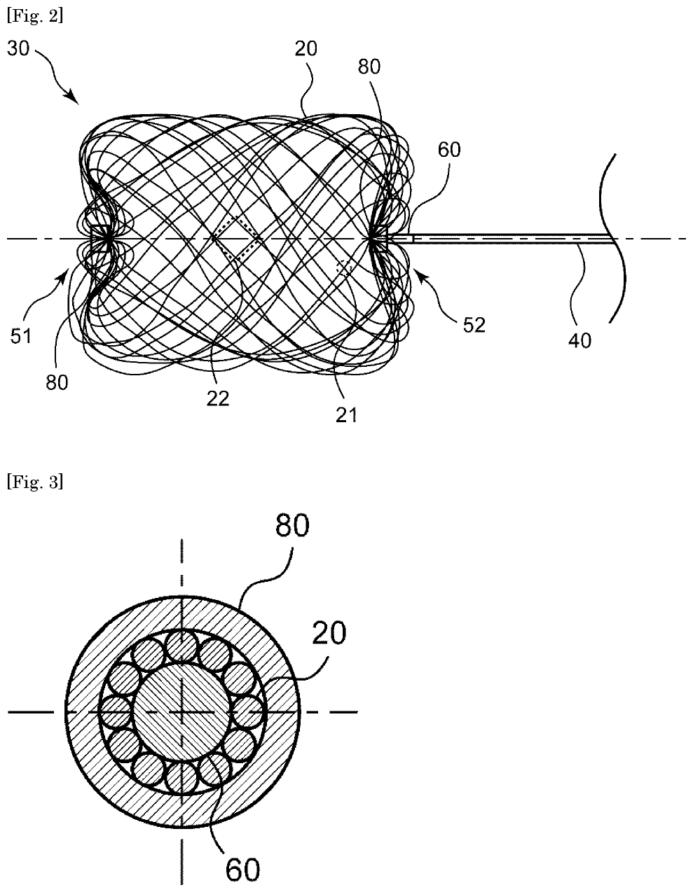

FIG. 1 is a cross-sectional view, of an embolization device 1 according to an embodiment of the present invention, parallel to the longitudinal direction of an outer tube 10, FIG. 2 is a plan view of a basket 30 of the embolization device 1, and FIG. 3 is a cross-sectional view, of the embolization device 1, perpendicular to the longitudinal direction of the outer tube 10.

As shown in FIG. 1, the embolization device 1 of the present invention is an embolization device 1 for a bump in a lumen, including: the outer tube 10 having a distal end and a proximal end; the basket 30 disposed in the lumen of the outer tube 10, having a plurality of wires 20, expandable when coming out of the outer tube 10, and composed of a mesh-like wall surface on which the plurality of wires 20 intersect; and a basket pusher 40 disposed on the proximal side of the basket 30. The basket 30 has a first bundling portion 51 at which the plurality of wires 20 are bundled and fixed on a distal side thereof, and a second bundling portion 52 at which the plurality of wires 20 are bundled and fixed on a proximal side thereof, the plurality of wires 20 exist inside the second bundling portion 52, and no passage for inserting a medical long object 100 in the direction from the proximal side toward the distal side of the basket 30 exists inside the second bundling portion 52.

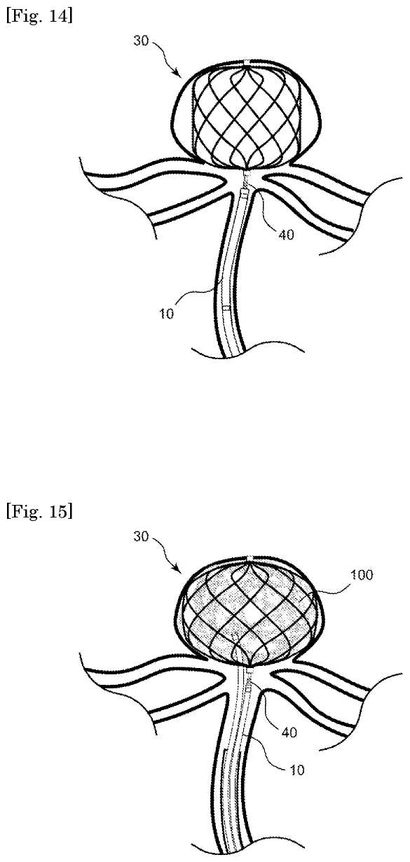

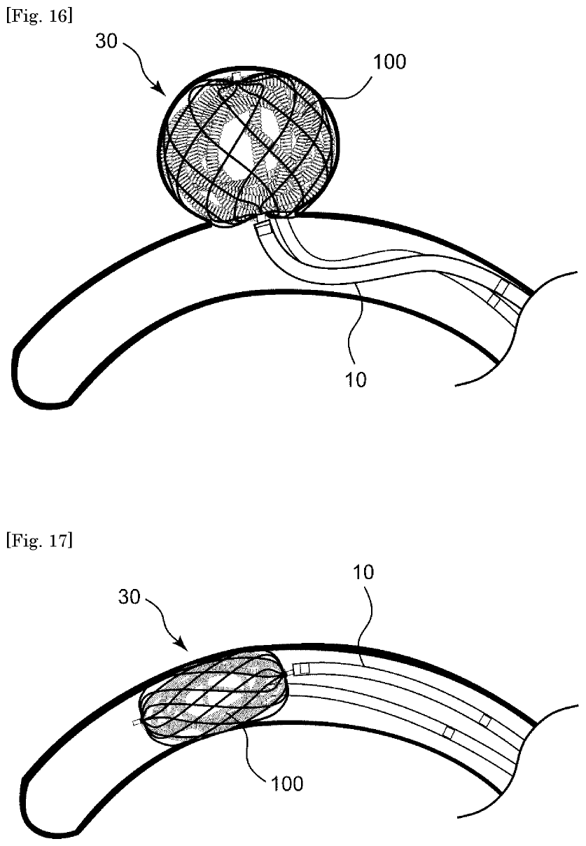

The embolization device 1 can be used in embolization which promotes formation of an embolus in a bump in a lumen such as an aneurysm in a blood vessel and prevents rupture of the bump. FIG. 14 is a schematic diagram of a state where the basket 30 is placed in a bump at a terminal portion of an in-vivo lumen, and FIG. 15 is a schematic diagram of a state where the medical long object 100 such as a coil is placed in the basket 30 shown in FIG. 14. Also, FIG. 16 is a schematic diagram of a state where the basket 30 and the medical long object 100 are placed in a bump at a side wall portion of an in-vivo lumen, and FIG. 17 is a schematic diagram of a state where the basket 30 and the medical long object 100 are placed in a main tube peripheral portion of an in-vivo lumen. As shown in FIG. 14 to FIG. 17, the embolization device 1 introduces the outer tube 10 into a lumen and indwells the basket 30 in a bump from the outer tube 10. Furthermore, the embolization device 1 places the medical long object 100 in the basket 30 indwelled in the bump and promotes formation of an embolus in the bump. As shown in FIG. 15 to FIG. 17, the embolization device 1 of the present invention allows the medical long object 100 to be inserted from the mesh portion of the wires 20 of the basket 30 of the embolization device 1.

As shown in FIG. 1, the outer tube 10 has a distal end and a proximal end. In the present invention, the proximal side refers to the hand side of a user with respect to a direction in which the outer tube 10 extends, and the distal side refers to a side opposite to the proximal side, that is, a side (lesion site side) on which treatment is performed with the embolization device 1. Also, the direction in which the outer tube 10 extends is referred to as a longitudinal direction. In other words, the longitudinal direction of the outer tube 10 is a distal-proximal direction of the outer tube 10. In FIG. 1 and FIG. 2, the right side of the drawing is the proximal side, and the left side of the drawing is the distal side.

The outer tube 10 is a tube-like member extending in the longitudinal direction, and has at least one lumen. The number of lumens of the outer tube 10 may be a plural number, but is preferably one. When the number of lumens of the outer tube 10 is one, the outer diameter of the outer tube 10 can be reduced, so that the embolization device 1 can be less invasive.

The material forming the outer tube 10 is preferably a resin or a metal. Examples of the resin forming the outer tube 10 include polyamide-based resins, polyester-based resins, polyurethane-based resins, polyolefin-based resins, fluorine-based resins, vinyl chloride-based resins, silicone-based resins, natural rubber, etc. Only one of these materials may be used, or two or more of these materials may be used in combination. Among them, the resin forming the outer tube 10 is preferably at least one of polyamide-based resins, polyester-based resins, polyurethane-based resins, polyolefin-based resins, and fluorine-based resins. When the material forming the outer tube 10 is at least one of polyamide-based resins, polyester-based resins, polyurethane-based resins, polyolefin-based resins, and fluorine-based resins, the slipperiness of the surface of the outer tube 10 can be enhanced, and the insertability of the outer tube 10 into a lumen such as a blood vessel can be improved. A resin tube forming the outer tube 10 is produced using a normal method such as extrusion molding and injection molding.

Examples of the metal forming the outer tube 10 include stainless steel such as SUS304 and SUS316, platinum, nickel, cobalt, chromium, titanium, tungsten, gold, Ni—Ti alloys, Co—Cr alloys, and combinations thereof. As a metal tube forming the outer tube 10, a tube obtained by spirally winding a metal wire material, a tube obtained by knitting a metal wire material, or the like may be used. In addition, the outer tube 10 may be a tube obtained by combining a metal and a resin. A tubular body formed from a resin and having a reinforcing material such as a metal wire material provided therein may be used as the outer tube 10. In the case where a tube-like member made of a resin and having a wire material provided therein is used as the outer tube 10, the wire material is preferably formed from a Ni—Ti alloy since the wire material has excellent shape memory and high elasticity. In addition to the above-described metals, the wire material may be a fiber material such as polyarylate fiber, aramid fiber, ultra-high molecular weight polyethylene fiber, PBO fiber, and carbon fiber. The fiber material may be monofilament or may be multifilament.

The outer tube 10 may be composed of a single layer or may be composed of a plurality of layers. In addition, in the longitudinal direction, a part of the outer tube 10 may be composed of a single layer, and the other part of the outer tube 10 may be composed of a plurality of layers.

The cross-sectional shape of the outer tube 10 in a cross-section perpendicular to the longitudinal direction may be a circular shape, an elliptical shape, a polygonal shape, or a combination thereof. In addition, the cross-sectional shape of the lumen of the outer tube 10 in the cross-section perpendicular to the longitudinal direction may also be a circular shape, an elliptical shape, a polygonal shape, or a combination thereof.

As shown in FIG. 1, the basket 30 is disposed in the lumen of the outer tube 10, has the plurality of wires 20, is expandable when coming out of the outer tube 10, and is composed of a mesh-like wall surface on which the plurality of wires 20 intersect. That is, the basket 30 is disposed in the lumen of the outer tube 10, and as shown in FIG. 2, the basket 30 can expand when released from the outer tube 10. When the basket 30 is disposed in the lumen of the outer tube 10, the basket 30 is in contact with an inner wall of the outer tube 10 and is squeezed by an external force received from the outer tube 10. When the basket 30 is released from the outer tube 10, the basket 30 does not receive the external force, and comes into an expanded state if no external force is applied thereto. When the basket 30 is placed into a bump, the basket 30 comes into contact with a wall portion of the bump and becomes deformed by an external force received from the bump.

Since the basket 30 is composed of the mesh-like wall surface on which the plurality of wires 20 intersect, the mesh of the basket 30 can be finer than that of a conventional embolization device. Therefore, in embolization of an aneurysm or the like, the medical long object 100, such as a coil, placed inside the basket 30 that is indwelled in the bump is less likely to come out of the basket 30 through an opening 22 of the mesh. As a result, the medical long object 100 can be stably placed inside the basket 30, that is, in the bump.

The material forming the wires 20 preferably has elasticity, and examples thereof include metal wire materials that are single wires or twisted wires formed from stainless steel such as SUS304 and SUS316, platinum, nickel, cobalt, chromium, titanium, tungsten, aluminum, gold, silver, Ni—Ti alloys, Co—Cr alloys, etc. Among them, the material forming the wires 20 is preferably a metal wire material of a Ni—Ti alloy. When the material forming the wires 20 is a metal wire material of a Ni—Ti alloy, the elasticity of each wire 20 can be enhanced, so that the basket 30 released from the outer tube 10 can be quickly expanded.

It is sufficient that the number of the wires 20 of the basket 30 is a plural number, and the number of wires 20 can be selected according to the inner diameter of an in-vivo lumen such as a blood vessel, etc. In the drawings, the number of the wires 20 of the basket 30 is limited. However, in an embodiment of the present invention, the basket 30 can be, for example, a basket 30 including 8 to 48 wires 20. The smaller the gaps of the mesh of the basket 30 are, the more likely it is to prevent the medical long object 100 from falling out. The wire diameter of each wire 20 can be set according to the size of the basket 30, the number and the material of the wires 20, etc. The number of the wires 20 of the basket 30 is preferably 16 to 32.

As shown in FIG. 1 and FIG. 2, the basket pusher 40 is disposed on the proximal side of the basket 30. The basket pusher 40 can move the basket 30 in the longitudinal direction of the outer tube 10 and release the basket 30 from the outer tube 10 by moving the basket pusher 40 in the longitudinal direction of the outer tube 10. Although not shown, the basket pusher 40 preferably includes a handle for controlling the position in the longitudinal direction or rotation thereof, on the proximal side with respect to the proximal end of the outer tube 10.

The material forming the basket pusher 40 is preferably a metal, and examples thereof include metals such as stainless steel, carbon steel, and nickel-titanium alloys. Among them, the material forming the basket pusher 40 is preferably stainless steel. When the material forming the basket pusher 40 is stainless steel, the rigidity of the basket pusher 40 is increased. As a result, the force applied to the basket pusher 40 can be efficiently transmitted to the basket 30, so that it is easier to perform the operation of releasing the basket 30 from the outer tube 10.

As shown in FIG. 1 and FIG. 2, the basket 30 has the first bundling portion 51 at which the plurality of wires 20 are bundled and fixed on the distal side thereof, and the second bundling portion 52 at which the plurality of wires 20 are bundled and fixed on the proximal side thereof. The basket 30 is formed in a cage shape having a mesh-like wall surface, for example, by combining a plurality of bent wires 20 between the first bundling portion 51 and the second bundling portion 52, or combining wires 20 twisted together in a right-handed spiral and wires 20 twisted together in a left-handed spiral.

Examples of a method for bundling and fixing the plurality of wires 20 at the first bundling portion 51 and the second bundling portion 52 include welding the plurality of wires 20, crimping the plurality of wires 20 using another member such as a bundling tool 80, adhering the plurality of wires 20 using an adhesive, and fixing the plurality of wires 20 with a brazing material. The bundling tool 80 will be described in detail later.

As shown in FIG. 3, the plurality of wires 20 exist inside the second bundling portion 52, and no passage for inserting the medical long object 100 in the direction from the proximal side toward the distal side of the basket 30 exists inside the second bundling portion 52. Examples of the medical long object 100 include coils, wires, string-like objects, etc. Furthermore, the medical long object 100 may be a tube for transporting a long object such as a coil. In addition, the tubular medical long object 100 may be for transporting a semi-solid fluid, a gel-like material, a semi-solid, a liquid, or the like, or a bag-like object into a bump. The fact that no passage for inserting the medical long object 100 exists inside the second bundling portion 52 indicates that the second bundling portion 52 has therein no opening having a size that allows the medical long object 100 to passes therethrough.

In the embolization device 1 of the present invention, since no passage for inserting the medical long object 100 in the direction from the proximal side toward the distal side of the basket 30 exists inside the second bundling portion 52, the medical long object 100 is inserted from the mesh-like wall surface, on which the plurality of wires 20 of the basket 30 intersect, instead of inserting the medical long object 100 into the basket 30 from a portion where proximal end portions of the wires 20 forming the basket 30 are bundled as in a conventional embolization device. As shown in FIG. 2, a plurality of openings 22 of the mesh exist in the mesh-like wall surface of the basket 30, so that an insertion opening for inserting the medical long object 100 can be selected according to the condition of a lesion site in which the basket 30 is indwelled, etc. Therefore, it can be easier to insert the medical long object 100 into the basket 30 in response to the conditions of various lesion sites.

As in the second bundling portion 52, preferably, the plurality of wires 20 exist inside the first bundling portion 51, and no passage for inserting the medical long object 100 in the direction from the distal side toward the proximal side of the basket 30 exists inside the first bundling portion 51. When no passage for inserting the medical long object 100 in the direction from the distal side toward the proximal side of the basket 30 exists inside the first bundling portion 51, the first bundling portion 51 can be thinner, and further, a release mechanism can be simplified. In addition, the fixing strength of the plurality of wires 20 at the first bundling portion 51 can be increased.

As shown in FIG. 2, the plurality of wires 20 of the basket 30 have intersection points 21. Preferably, the intersection points 21 of the wires 20 are not fixed. That is, preferably, at each intersection point 21, each wire 20 is not fixed. When the intersection points 21 of the wires 20 are not fixed, each wire 20 can move when an external force is applied to the basket 30. As a result, when passing the medical long object 100 such as a coil through the mesh-like wall surface on which the plurality of wires 20 of the basket 30 intersect, the wires 20 can be moved by an external force and the size of the openings 22 of the mesh of the basket 30 can be increased, so that it is easier to place the medical long object 100 in the basket 30. In addition, when the intersection points 21 of the wires 20 are not fixed, it is easier for the basket 30 to become deformed when placing the medical long object 100 inside the basket 30. Therefore, it is also possible for the basket 30 to become deformed so as to match the shape of a bump, thereby promoting generation of an embolus having a size or shape that fills the inside of the bump and enhancing the effect of preventing rupture of the bump.

In the basket 30, preferably, the positions of the intersection points 21 of the wires 20 of the mesh are shifted by an external force, and the shapes of the openings 22 of the mesh are changed. Specifically, for example, when inserting the medical long object 100 through the wall surface of the basket 30, one opening 22 having a diamond shape as shown in FIG. 2 is expanded into a square-like shape by an external force. When the shape of the opening 22 of the mesh of the basket 30 is changed by an external force, it is easier for the medical long object 100 to be inserted through the opening 22 of the mesh, so that it is easier to perform the operation of placing the medical long object 100 in the basket 30.

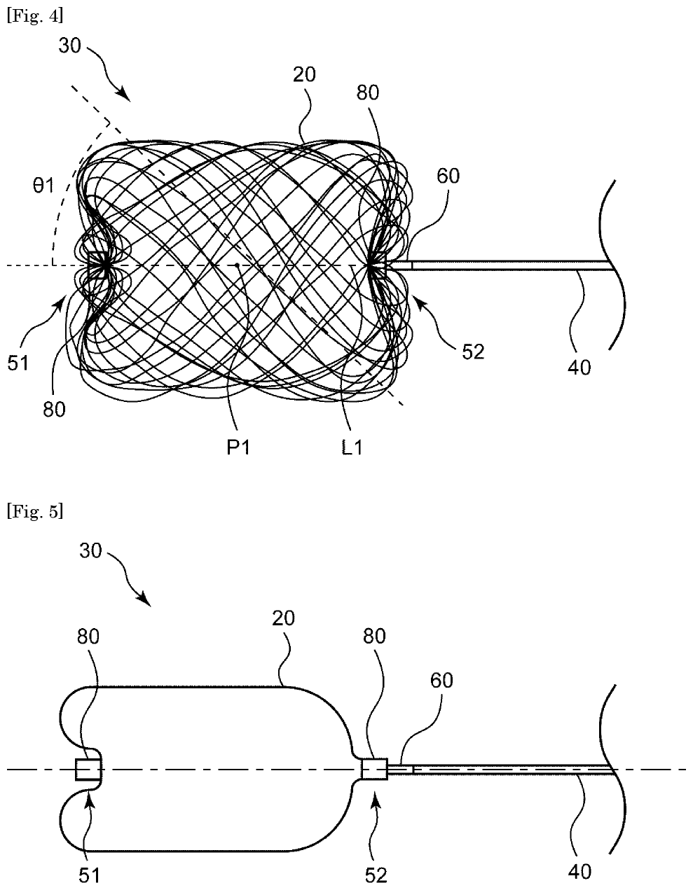

FIG. 4 is a plan view of the basket 30 of the embolization device 1. In FIG. 4, the right side of the drawing is the proximal side, and the left side of the drawing is the distal side. As shown in FIG. 4, in a state where no external force is applied to the basket 30, the basket 30 viewed in a direction perpendicular to the longitudinal direction of the outer tube 10 preferably has an opening 22 of the mesh in which an angle θ1 formed on the distal side between a straight line L1 passing through the first bundling portion 51 and the second bundling portion 52 and the wire 20 at a midpoint P1 of a straight line connecting the first bundling portion 51 and the second bundling portion 52 is equal to or larger than 30 degrees and smaller than 90 degrees. That is, the angle θ1 formed between the straight line L1 and the wire 20 forming the opening 22 of the mesh overlapping the midpoint P1 as viewed in the direction perpendicular to the longitudinal direction of the outer tube 10 is preferably equal to or larger than 30 degrees and smaller than 90 degrees. When the basket 30 has the opening 22 of the mesh in which the angle θ1 formed on the distal side between the straight line L1 and the wire 20 is equal to or larger than 30 degrees and smaller than 90 degrees, it is easier for the medical long object 100 to pass through the opening 22 of the mesh and it is difficult for the medical long object 100 placed inside the basket 30 to pass through the opening 22 of the mesh. As a result, the medical long object 100 can be stably indwelled at a target site such as a lesion site.

Moreover, in a state where no external force is applied to the basket 30, the basket 30 viewed in the direction perpendicular to the longitudinal direction of the outer tube 10 preferably also has an opening 22 of the mesh in which an angle in the axial direction at the intersection point 21 of the wires 20 is not smaller than 60 degrees and not larger than 160 degrees. When the basket 30 has the opening 22 of the mesh in which in the angle in the axial direction at the intersection point 21 of the wires 20 is not smaller than 60 degrees and not larger than 160 degrees, it is easier to increase the area of the opening 22 of the mesh, so that it is easier for the medical long object 100 to pass through the opening 22 of the mesh. As a result, it is easier to place the medical long object 100 in the basket 30.

Preferably, the basket 30 is deformable and is slidable inside a tube having an inner diameter of 0.021 inch or smaller. In the case where the number of lumens of the outer tube 10 is one, preferably, the inner diameter of the outer tube 10 is not larger than 0.021 inch, and the basket 30 is disposed in the lumen of the outer tube 10. In addition, in the case where the outer tube 10 has a plurality of lumens, the inner diameter of the lumen in which the basket 30 is disposed is preferably not larger than 0.021 inch. When the basket 30 is deformable and is slidable inside a tube having an inner diameter of 0.021 inch or smaller, the outer diameter of the outer tube 10 can be reduced. As a result, the embolization device 1 can have good insertability and be less invasive.

As shown in FIG. 1, FIG. 2, and FIG. 4, the embolization device 1 preferably has a severable connection member 60 which is disposed on the proximal side with respect to the basket 30 and on the distal side with respect to the basket pusher 40. The connection member 60 is a member connecting the basket 30 and the basket pusher 40, and is severable. When the embolization device 1 has the connection member 60, the basket 30 is detached from the basket pusher 40 by severing the connection member 60 after the basket 30 is transported to a target site, so that it is possible to easily indwell the basket 30 at the target site.

As a method for severing the connection member 60, various methods using a mechanical severing mechanism, thermal cutting, thermal, electrical, and chemical severing, etc., can be used. Examples of the connection member 60 include rod-like objects, string-like objects, clips, members fitted to each other such as recesses and projections, etc. As the material forming the connection member 60, synthetic resins, metals, etc., can be used. The connection member 60 may be a member different from the basket 30 or the basket pusher 40, or may be a part of the basket 30 or the basket pusher 40.

Preferably, the material forming the connection member 60 has a property of melting due to heat, and the embolization device 1 has a heating mechanism 70 for heating the connection member 60. When the material forming the connection member 60 has a property of melting due to heat and the embolization device 1 has the heating mechanism 70 for heating the connection member 60, the connection member 60 can be melted and broken by the heating mechanism 70 heating the connection member 60, so that the basket 30 can be detached from the basket pusher 40. Therefore, the basket 30 and the basket pusher 40 can be firmly connected by the connection member 60 until the heating mechanism 70 is activated, and the connection member 60 can be easily severed when the heating mechanism 70 is activated. Thus, the basket 30 can be reliably and easily indwelled.

As the material forming the connection member 60 and having a property of melting due to heat, thermoplastic resins are preferable, and among them, PVA (polyvinyl alcohol) is more preferable. When the material for the connection member 60 is PVA, the connection member 60 can be more easily severed, so that the embolization device 1 can be easily handled.

The heating mechanism 70 is preferably connected to the basket pusher 40. When the heating mechanism 70 is connected to the basket pusher 40, the connection member 60 can be heated via the basket pusher 40, so that it is not necessary to additionally provide a member for transmitting the heat of the heating mechanism 70 to the connection member 60 and the size of the embolization device 1 can be reduced.

In a cross-section perpendicular to the longitudinal direction of the outer tube 10, preferably, as shown in FIG. 3, the connection member 60 is disposed inside the second bundling portion 52, and the wires 20 are disposed outside the connection member 60. That is, preferably, the connection member 60 is disposed at a center portion of the second bundling portion 52 in the cross-section perpendicular to the longitudinal direction of the outer tube 10, and the plurality of wires 20 are disposed outside the connection member 60. When, in the cross-section perpendicular to the longitudinal direction of the outer tube 10, the connection member 60 is disposed inside the second bundling portion 52 and the wires 20 are disposed outside the connection member 60, the contact area between the connection member 60 and the wires 20 can be increased. Therefore, the strength of the connection between the connection member 60 and the basket 30 is increased, so that the basket 30 is less likely to be unintentionally detached from the connection member 60 during transport of the basket 30.

FIG. 5 to FIG. 13 are schematic diagrams of various baskets 30 of embolization devices 1 according to embodiments of the present invention. In FIG. 5 to FIG. 13, for facilitating understanding of the positional relationship between the first bundling portion 51, the second bundling portion 52, and the distal end and the proximal end of the basket 30, only some of the wires 20 are shown, and the other wires 20 are not shown.

As shown in FIG. 5 to FIG. 10, the first bundling portion 51 is preferably located on the proximal side with respect to the distal end of the basket 30 in a state where no external force is applied to the basket 30. Since the first bundling portion 51 and the second bundling portion 52 are portions at which the plurality of wires 20 are bundled and fixed, the first bundling portion 51 and the second bundling portion 52 have higher rigidity than the other portion of the basket 30. When the first bundling portion 51 is located on the proximal side with respect to the distal end of the basket 30, a distal end portion of the basket 30 is flexible. As a result, when transporting the basket 30 to a target site or indwelling the basket 30 at the target site, even if the distal end portion of the basket 30 comes into contact with a wall portion of an in-vivo lumen such as a blood vessel, the wall portion of the in-vivo lumen can be less likely to be damaged, so that the safety of the embolization device 1 can be enhanced.

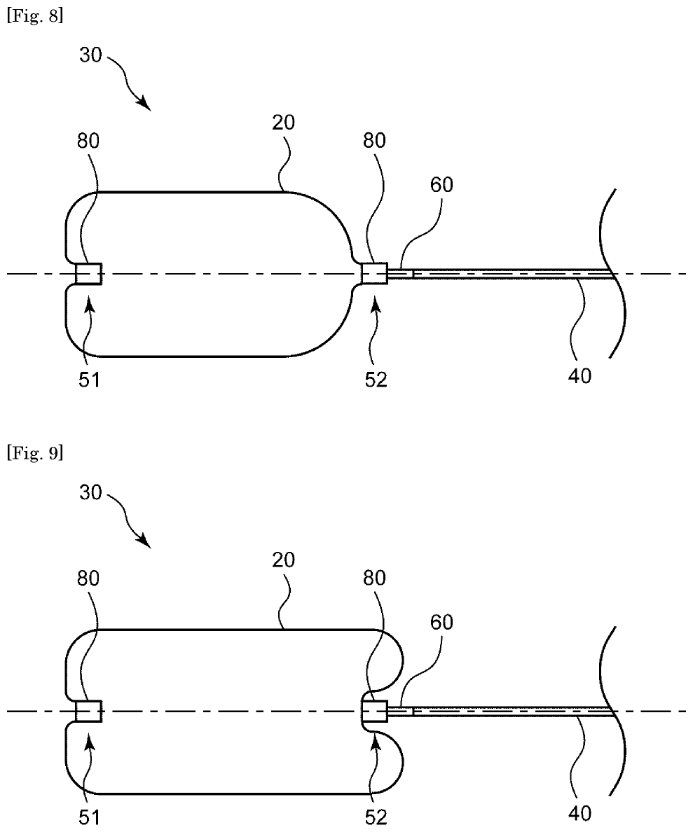

In the case where the first bundling portion 51 is located on the proximal side with respect to the distal end of the basket 30, as shown in FIG. 5 to FIG. 7, the wires 20 may exist on the proximal side of the first bundling portion 51 and the distal end portion of the basket 30 may have a recess shape, or as shown in FIG. 8 to FIG. 10, the wires 20 may exist on the distal side of the first bundling portion 51 and the first bundling portion 51 may be located inside the basket 30.

As shown in FIG. 11 to FIG. 13, the first bundling portion 51 may be located on the distal side with respect to the distal end of the basket 30 in a state where no external force is applied to the basket 30. When the first bundling portion 51 is located on the distal side with respect to the distal end of the basket 30, the first bundling portion 51 having higher rigidity than the other portion of the basket 30 is located at a most distal portion of the basket 30. Therefore, the insertability of the basket 30 is increased, so that it is easier to send the basket 30 to a target site.

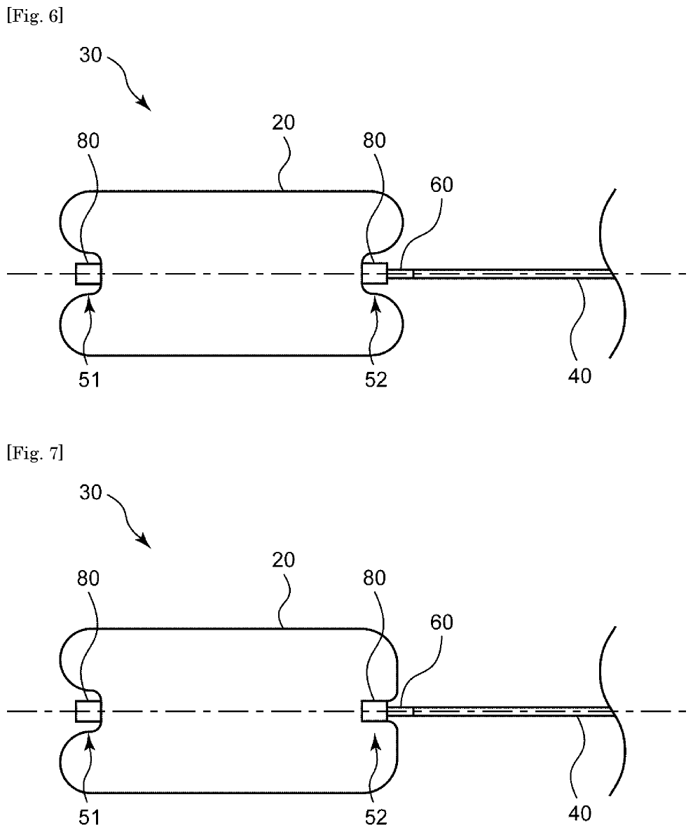

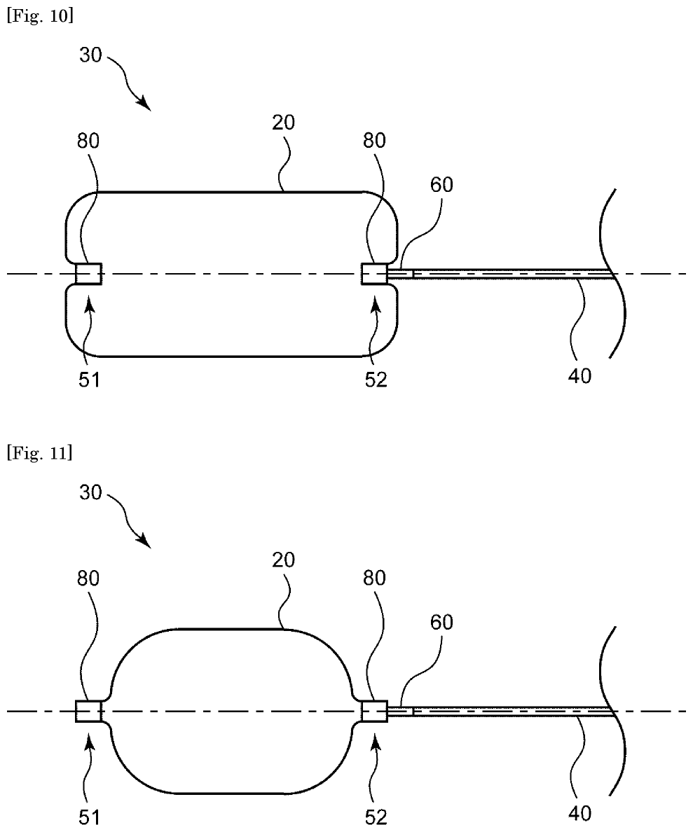

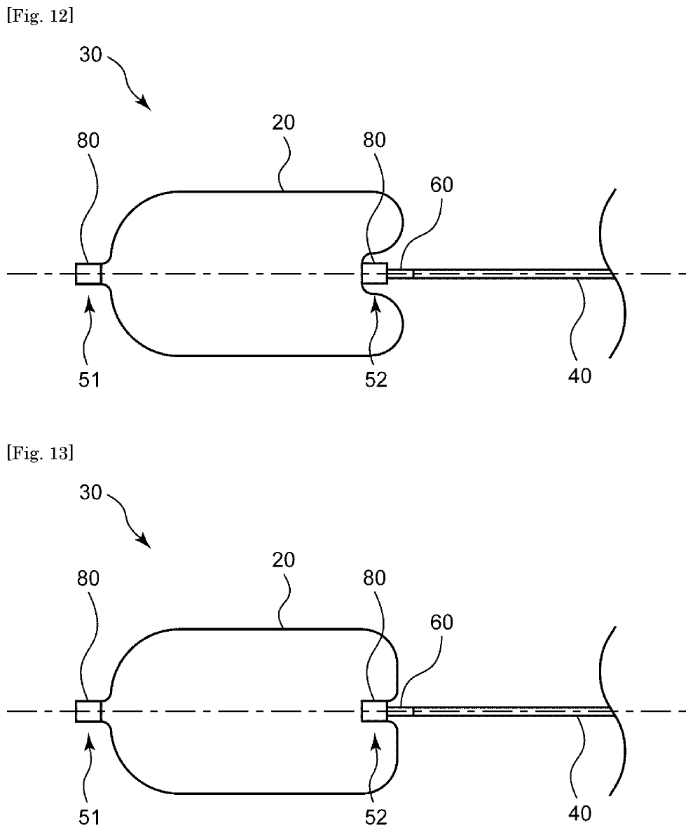

As shown in FIG. 6, FIG. 7, FIG. 9, FIG. 10, FIG. 12, and FIG. 13, the second bundling portion 52 is preferably located on the distal side with respect to the proximal end of the basket 30 in a state where no external force is applied to the basket 30. When the second bundling portion 52 is located on the distal side with respect to the proximal end of the basket 30, the surface on the proximal side of the basket 30 is smooth. Therefore, even if the proximal side of the basket 30 comes into contact with an inner wall of a blood vessel or the like during transport or indwelling of the basket 30, the inner wall of the blood vessel or the like is less likely to be damaged, so that the embolization device 1 can be highly safe.

In the case where the second bundling portion 52 is located on the distal side with respect to the proximal end of the basket 30, as shown in FIG. 6, FIG. 9, and FIG. 12, the wires 20 may exist on the distal side of the second bundling portion 52 and a proximal end portion of the basket 30 may have a recess shape, or as shown in FIG. 7, FIG. 10, and FIG. 13, the wires 20 may exist on the proximal side of the second bundling portion 52 and the second bundling portion 52 may be located inside the basket 30.

As shown in FIG. 5, FIG. 8, and FIG. 11, the second bundling portion 52 is preferably located on the proximal side with respect to the proximal end of the basket 30 in a state where no external force is applied to the basket 30. When the second bundling portion 52 is located on the proximal side with respect to the proximal end of the basket 30 in the lumen of the outer tube 10, a force is easily transmitted to the basket 30 when the basket pusher 40 is moved to the distal side in the outer tube 10. Therefore, it is possible to quickly release the basket 30 from the outer tube 10.

As shown in FIG. 5 to FIG. 10, the first bundling portion 51 is preferably located on the proximal side with respect to the distal end of the basket 30 in the lumen of the outer tube 10. When the first bundling portion 51 is located on the proximal side with respect to the distal end of the basket 30 in the lumen of the outer tube 10, the first bundling portion 51 does not exist on the distal side with respect to the distal end of the basket 30, and the outer surface of the basket 30 is smooth. As a result, the friction generated between the outer surface on the distal side of the basket 30 and the inner surface of the outer tube 10 can be reduced, and the first bundling portion 51 is less likely to get caught on another object, so that the basket 30 can be smoothly released from the outer tube 10.

As shown in FIG. 11 to FIG. 13, the first bundling portion 51 may be located on the distal side with respect to the distal end of the basket 30 in the lumen of the outer tube 10. When the first bundling portion 51 is located on the distal side with respect to the distal end of the basket 30 in the lumen of the outer tube 10, contact between the first bundling portion 51 and the wires 20 of the basket 30 can be avoided. Therefore, the basket 30 can be smoothly deployed in a bump.

As shown in FIG. 6, FIG. 7, FIG. 9, FIG. 10, FIG. 12, and FIG. 13, the second bundling portion 52 is preferably located on the distal side with respect to the proximal end of the basket 30 in the lumen of the outer tube 10. When the second bundling portion 52 is located on the distal side with respect to the proximal end of the basket 30 in the lumen of the outer tube 10, the second bundling portion 52 does not exist on the proximal side with respect to the proximal end of the basket 30. Therefore, the outer surface on the proximal side of the basket 30 is smooth, so that another object can be prevented from coming into contact with the proximal side of the basket 30 and shifting the position of the basket 30 after the basket 30 is indwelled at a target site.

As shown in FIG. 5, FIG. 8, and FIG. 11, the second bundling portion 52 is preferably located on the proximal side with respect to the proximal end of the basket 30 in the lumen of the outer tube 10. When the second bundling portion 52 is located on the proximal side with respect to the proximal end of the basket 30 in the lumen of the outer tube 10, contact between the second bundling portion 52 and the wires 20 of the basket 30 can be avoided, so that the basket 30 can be smoothly deployed in a bump.

As shown in FIG. 1, FIG. 2, and FIG. 4, at least one of the first bundling portion 51 and the second bundling portion 52 is preferably bundled by the bundling tool 80. When the at least one of the first bundling portion 51 and the second bundling portion 52 is bundled by the bundling tool 80, the plurality of wires 20 can be reliably bundled and fixed.

Examples of the bundling tool 80 include a ring-shaped member, a member obtained by forming a slit in a ring and having a C-shaped cross-section, a coil-like member obtained by winding a wire material, and a member for tying and fixing with a string-like object. Among them, the bundling tool 80 is preferably a ring-shaped member. As for the bundling tool 80 that is a ring-shaped member, for example, the first bundling portion 51 or the second bundling portion 52 can be formed by passing the plurality of wires 20 through the inner cavity of the bundling tool 80 and crimping the bundling tool 80. When the bundling tool 80 is a ring-shaped member, the plurality of wires 20 can be firmly fixed by the bundling tool 80, so that the plurality of wires 20 can be less likely to be de-bundled.

As the material forming the bundling tool 80, for example, the same materials as those for the wires 20 of the basket 30 and the basket pusher 40 can be used. Among them, the material forming the bundling tool 80 is preferably stainless steel. When the material forming the bundling tool 80 is stainless steel, the fixing strength of the plurality of wires 20 can be enhanced, and the durability of the bundling tool 80 can be improved.

The bundling tool 80 also preferably contains an X-ray impermeable material. An example of the X-ray impermeable material is at least one material selected from the group consisting of lead, barium, iodine, tungsten, gold, platinum, iridium, platinum-iridium alloys, stainless steel, titanium, cobalt-chromium alloys, palladium, and tantalum. When the bundling tool 80 contains the X-ray impermeable material, the position of the bundling tool 8080 can be confirmed under X-ray fluoroscopy. As a result, it is possible to grasp the position of the basket 30 in a body.

Preferably, the wires 20 of the basket 30 include a first wire and a second wire, and the material forming the first wire is different from the material forming the second wire. When the wires 20 include the first wire and the second wire which are formed from different materials, it is possible to impart physical properties, such as rigidity and elasticity, that are difficult to be achieved with one type of material, to the basket 30 by, for example, forming the first wire using a material having high rigidity and forming the second wire using a material having high elasticity. In addition, the first wire may be formed using a material having high elasticity, and the second wire may be formed using a material having high X-ray impermeability. When the first wire is formed using a material having high elasticity and the second wire is formed using a material having high X-ray impermeability, the basket 30 can be visually recognized under X-ray fluoroscopy, and the basket 30 can have excellent elasticity.

The second wire is preferably composed of a structure obtained by twisting together a plurality of wire materials. When the second wire is composed of a structure obtained by twisting together a plurality of wire materials, for example, even if the material forming the second wire is a material having inferior strength or elasticity, it is possible to improve the strength or elasticity of the second wire by forming the second wire as a twisted wire.

In the case where the second wire is composed of a structure obtained by twisting together a plurality of wire materials, the first wire is preferably a single wire. That is, the basket 30 preferably has a first wire that is a single wire, and a second wire that is a twisted wire. When the second wire has a structure obtained by twisting together a plurality of wire materials and the first wire is a single wire, various characteristics can be imparted to the basket 30 by, for example, using a material having a characteristic that the first wire does not have but having insufficient strength or elasticity in the form of a single wire, for the second wire, and using a material having sufficient strength or elasticity in the form of a single wire, for the first wire.

The second wire preferably contains an X-ray impermeable material which is, for example, one material selected from the group consisting of lead, barium, iodine, tungsten, gold, platinum, iridium, platinum-tungsten alloys, platinum-iridium alloys, stainless steel, titanium, cobalt-chromium alloys, palladium, and tantalum, and the second wire is preferably composed of a structure obtained by twisting together a plurality of wire materials. The X-ray impermeable material normally has low elasticity and has high rigidity and poor plastic deformation. By using a wire material containing the X-ray impermeable material to form the basket 30 that can expand when coming out of the outer tube 10, it is necessary to reduce the thickness of the wire material containing the X-ray impermeable material. On the other hand, when the wire material is thinned, the visibility under X-ray fluoroscopy decreases. Therefore, when the second wire contains the X-ray impermeable material and is composed of a twisted wire, it is possible to achieve both elasticity of the second wire and visibility under X-ray fluoroscopy.

Although not shown, at least one of the first wire and the second wire also preferably has a configuration of having a core portion and an outer layer portion covering the core portion. Specifically, the first wire may have a configuration of having a core portion and an outer layer portion and the second wire may have a single-layer structure having no core portion and no outer layer portion, or the first wire may have a single-layer structure and the second wire may have a configuration of having a core portion and an outer layer portion, or the first wire and the second wire may each have a configuration of having a core portion and an outer layer portion, or the first wire and the second wire may each have a single-layer structure.

Examples of the materials forming the core portion and the outer layer portion of at least one of the first wire and the second wire include stainless steel such as SUS304 and SUS316, and metals such as platinum, nickel, cobalt, chromium, titanium, tungsten, aluminum, gold, silver, lead, barium, iodine, iridium, stainless steel, titanium, Ni—Ti alloys, and Co—Cr alloys. Among them, the first wire and the second wire preferably each have a configuration having a core portion containing an X-ray impermeable material and an outer layer portion containing a Ni—Ti alloy. When at least one of the first wire and the second wire has a configuration having a core portion containing an X-ray impermeable material and an outer layer portion containing a Ni—Ti alloy, it is possible to enhance the elasticity of the basket 30 by the outer layer portion to quickly expand the basket 30 released from the outer tube 10, while enhancing the visibility of the basket 30 under X-ray fluoroscopy by the core portion. In addition, the first wire and the second wire may have the same configuration.

As described above, the embolization device of the present invention is an embolization device for a bump in a lumen, including: an outer tube having a distal end and a proximal end; a basket disposed in a lumen of the outer tube, having a plurality of wires, expandable when coming out of the outer tube, and composed of a mesh-like wall surface on which the plurality of wires intersect; and a basket pusher disposed on a proximal side of the basket, wherein the basket has a first bundling portion at which the plurality of wires are bundled and fixed on a distal side thereof and a second bundling portion at which the plurality of wires are bundled and fixed on the proximal side thereof, the plurality of wires exist inside the second bundling portion, and no passage for inserting a medical long object in a direction from the proximal side toward the distal side of the basket exists inside the second bundling portion. Since the embolization device of the present invention is configured as described above, it is easier to indwell the medical long object at a lesion site, and it can be difficult for the medical long object placed inside the basket to come out of the basket.

This application claims priority to Japanese Patent Application No. 2020-213880, filed on Dec. 23, 2020. All of the contents of the Japanese Patent Application No. 2020-213880, filed on Dec. 23, 2020, are incorporated by reference herein.

### REFERENCE SIGNS LIST

1: embolization device
10: outer tube
20: wire
21: intersection point
22: opening
30: basket
40: basket pusher
51: first bundling portion
52: second bundling portion
60: connection member
70: heating mechanism
80: bundling tool
100: medical long object
P1: midpoint of a straight line connecting the first bundling portion and the second bundling portion
L1: straight line passing through the first bundling portion and the second bundling portion
θ1: an angle formed on the distal side between a straight line passing through the first bundling portion and the second bundling portion and the wire

1. An embolization device for a bump in a lumen, comprising:
an outer tube having a distal end and a proximal end;
a basket comprising a plurality of wires, the plurality of wires configured to form a mesh structure having a meshed wall surface in which the plurality of wires intersect each other; and
a basket pusher disposed on a proximal side of the basket, wherein,
the basket is disposed in a lumen of the outer tube so that the basket is expandable when the basket comes out of the outer tube,
the basket has a first bundling portion and a second bundle portion, and distal ends of the plurality of wires are bundled and fixed at the first bundling portion on a distal side of the basket and proximal ends of the plurality of wires are bundled and fixed at the second bundling portion on the proximal side of the basket, and
the plurality of wires are disposed inside the second bundling portion so that no passage for inserting a medical long object in a direction from the proximal side toward the distal side of the basket exists inside the second bundling portion.

2. The embolization device according to claim 1, wherein the plurality of wires are not fixed to each other at an intersection point of the meshed wall surface.

3. The embolization device according to claim 1, wherein the basket is configured so that a position of an intersection point of the plurality of the wires of the meshed wall surface is shifted by an external force and a shape of an opening of the meshed wall surface is changed.

4. The embolization device according to claim 1, wherein, in a state where no external force is applied to the basket and the basket is fully expanded, the basket viewed in a direction perpendicular to a longitudinal direction of the outer tube has an opening of the meshed wall surface, in which an angle formed on the distal side between a straight line passing through the first bundling portion and the second bundling portion and the wire at a midpoint between the first bundling portion and the second bundling portion is equal to or larger than 30 degrees and smaller than 90 degrees.

5. The embolization device according to claim 1, wherein, in a state where no external force is applied to the basket and the basket is fully expanded, the basket viewed in the direction perpendicular to the longitudinal direction of the outer tube has an opening of the meshed wall surface, in which an angle formed in an axial direction at the intersection point of the wires is not smaller than 60 degrees and not larger than 160 degrees.

6. The embolization device according to claim 1, wherein the basket is deformable and is slidable inside a tube having an inner diameter of 0.021 inch or smaller.

7. The embolization device according to claim 1, further comprising a detachable connection member disposed between the basket and the basket pusher.

8. The embolization device according to claim 7, further comprising a heater for heating the detachable connection member, wherein
the detachable connection member comprises a material having a property of melting due to heat generated by the heater.

9. The embolization device according to claim 7,
wherein in a cross-section perpendicular to the longitudinal direction of the outer tube, the detachable connection member is disposed inside the second bundling portion, and
the wires are disposed outside the connection member.

10. The embolization device according to claim 1, wherein, in a state where no external force is applied to the basket and the basket is fully expanded, the first bundling portion is located on the proximal side with respect to a distal end of the basket.

11. (canceled)

12. The embolization device according to claim 1, wherein, in a state where no external force is applied to the basket and the basket is fully expanded, the second bundling portion is located on the distal side with respect to a proximal end of the basket.

13. The embolization device according to claim 1, wherein, in a state where no external force is applied to the basket and the basket is fully expanded, the second bundling portion is located on the proximal side with respect to a proximal end of the basket.

14. The embolization device according to claim 1, wherein the basket is disposed in the lumen of the outer tube so that the first bundling portion is located on the proximal side with respect to the distal end of the basket in the lumen of the outer tube.

15. The embolization device according to claim 1, wherein the basket is disposed in the lumen of the outer tube so that the second bundling portion is located on the distal side with respect to the proximal end of the basket in the lumen of the outer tube.

16. The embolization device according to claim 1, wherein at least one of the first bundling portion and the second bundling portion is bundled by a bundling member.

17. The embolization device according to claim 1, wherein
the wires include a first wire and a second wire, and
a material forming the first wire is different from a material forming the second wire.

18. The embolization device according to claim 17, wherein at least one of the first wire and the second wire has a configuration having a core portion containing an X-ray impermeable material and an outer layer portion containing a Ni—Ti alloy.

19. The embolization device according to claim 18, wherein the second wire is composed of a structure obtained by twisting together a plurality of wire materials.

20. The embolization device according to claim 1, wherein, in a state where no external force is applied to the basket and the basket is fully expanded, the first bundling portion is located on the proximal side with respect to a distal end of the basket, and in a state where no external force is applied to the basket and the basket is fully expanded, the second bundling portion is located on the proximal side with respect to a proximal end of the basket.

21. The embolization device according to claim 1, wherein the plurality of wires are not fixed to each other at an intersection point of the meshed wall surface,
the basket is configured so that a position of an intersection point of the plurality of the wires of the meshed wall surface is shifted by an external force and a shape of an opening of the meshed wall surface is changed,
in a state where no external force is applied to the basket and the basket is fully expanded, the basket viewed in a direction perpendicular to a longitudinal direction of the outer tube has an opening of the meshed wall surface, in which an angle formed on the distal side between a straight line passing through the first bundling portion and the second bundling portion and the wire at a midpoint between the first bundling portion and the second bundling portion is equal to or larger than 30 degrees and smaller than 90 degrees, and
in a state where no external force is applied to the basket and the basket is fully expanded, the basket viewed in the direction perpendicular to the longitudinal direction of the outer tube has an opening of the meshed wall surface, in which an angle formed in an axial direction at the intersection point of the wires is not smaller than 60 degrees and not larger than 160 degrees.
